(19) United States
(12) Patent Application Publication
(10) Pub. No.: US 2013/0172821 A1
(43) Pub. Date: Jul. 4, 2013

(54) CATHETER INSERTION DEVICES

(71) Applicant: Daniel J. Potter, Stillwater, MN (US)

(72) Inventor: Daniel J. Potter, Stillwater, MN (US)

(21) Appl. No.: 13/340,086

(22) Filed: Dec. 29, 2011

(51) Int. Cl. A61M 25/01

(52) U.S. Cl. 604/171

(57) ABSTRACT

A catheter insertion device is provided that comprises an elongate member defining a longitudinal axis. The elongate member is configured to be disposed on a catheter in one of a first configuration and a second configuration. The first configuration and the second configuration are different. The catheter insertion device is further configured to allow the elongate member to be selectively and repeatedly reconfigured between the first configuration and the second configuration while disposed on the catheter.

### BACKGROUND OF THE INVENTION

a. Field of the Invention

The instant disclosure relates generally to catheter insertion devices. More particularly, the instant disclosure relates to reusable catheter insertion devices that can be used to insert a circular catheter, a balloon catheter, or other curved, complex, and/or fragile catheter into a hemostasis valve while allowing for additional usable length of the shaft of the catheter to be available when needed.

b. Background Art

The use of introducer catheters, balloon catheters, dilatation catheters, and other similar devices in a variety of diagnostic, therapeutic, and/or mapping and ablation procedures to diagnose and/or correct various cardiac conditions are well known. Often an introducer catheter or sheath can be used to guide a circular catheter, a balloon catheter, or other catheter. The introducer can include a hemostasis valve at its proximal end for preventing blood loss when the introducer is placed in an artery. Since less blood is lost with a hemostasis valve, the need for blood transfusions may be reduced. Although the hemostasis valve provides advantages, it can be difficult to insert a catheter through the hemostasis valve without damage to the catheter due to the curved, flexible, and/or fragile nature of the catheter. In particular, the tip of the catheter may be bent or curved to a preselected angle for purposes of insertion into an artery or vein, for example. When the catheter is inserted through the hemostasis valve, the bend in the tip of the catheter may change, making it difficult to properly position the catheter. Because it has been found to be difficult, if not impossible, to thread the flexible tip of a curved catheter through and past the hemostasis valve without impairing the predetermined bend in the tip, there is a need for a device and method which facilitates negotiating the catheter past the hemostasis valve during insertion of the catheter into the introducer.

Solid catheter straighteners may be used to facilitate insertion of a catheter into a hemostasis valve of an introducer. Solid catheter straighteners may comprise a solid, polymeric tube placed over a portion of the catheter. However, solid catheter straighteners cannot be removed from the catheter while in use as the catheter includes a handle at its proximal end that prevents removal of the solid catheter straightener from the catheter's proximal end and the catheter's distal end may be positioned inside a patient's body. The inability to remove the solid catheter straightener from the catheter may be disadvantageous when additional catheter length may be needed to access all cardiac chamber areas for a portion of the patient population that requires longer catheter reach because of longer, taller, and/or larger patient size/anatomy. Thus, the solid catheter straightener can result in a loss of usable length of the shaft of the catheter.

Some catheter straighteners include a helically or spirally cut tubular member and a pull member attached to the tubular member as provided in U.S. Pat. No. 4,569,347 to Frisbie, which is hereby incorporated by reference as though fully set forth herein. The pull member may be provided on one end of the tubular member to facilitate removal of the catheter straightener from the hemostasis valve of the introducer. However, such a catheter straightener requires the use of a mandrel or other device to make the catheter insertion device sufficiently rigid for insertion through the hemostasis valve, which must then be withdrawn and which adds to the complexity of the catheter straightener.

Other catheter insertion devices designed to compensate for the loss of usable length of the shaft of the catheter include splittable catheter straighteners or sheaths. Splittable catheter straighteners or sheaths can be formed having longitudinally extending scored lines of weakness along which the splittable catheter straightener or sheath can be split, peeled apart, and removed from the catheter. However, once the splittable catheter straightener or sheath has been removed by splitting it away from the shaft of the catheter, it cannot be reused. Accordingly, if the catheter is removed and then reinsertion of the catheter becomes necessary, a new splittable catheter straightener or sheath must be reloaded over the distal end of the catheter, which can be difficult and time-consuming. In addition, in some instances, no additional splittable catheter straightener or sheath may be immediately available.

There is therefore a need to minimize and/or eliminate one or more of the problems as set forth above.

### BRIEF SUMMARY OF THE INVENTION

It is desirable to provide a catheter insertion device that is reusable. As used herein, “reusable” refers to a medical device that may be reused during a single medical session for a single patient. A reusable catheter insertion device can potentially reduce procedure time and improve device durability by eliminating the need to reload the catheter insertion device over the distal end of the catheter if the catheter insertion device has been removed, as well as enhance device versatility by increasing the usable effective length of the shaft of the catheter. In particular, it may be desirable for the catheter insertion device to be completely removable from the catheter to increase the usable effective length of the shaft of the catheter in some embodiments. It may also be desirable for the catheter insertion device to be retractable on the catheter to increase the usable effective length of the shaft of the catheter in some embodiments.

A catheter insertion device is provided comprising an elongate member defining a longitudinal axis. The elongate member is configured to be disposed on a catheter in one of a first configuration and a second configuration. The first configuration and the second configuration are different. The catheter insertion device is configured to allow movement between the first configuration and the second configuration while disposed on the catheter. For example, the catheter insertion device can be configured to allow the elongate member to be selectively and repeatedly reconfigured between the first configuration and the second configuration while disposed on the catheter.

In accordance with a first embodiment of the disclosure, the elongate member can comprise a slit extending along the elongate member for an entire length of the elongate member. The slit can be disposed at a first circumferential location of the elongate member. The elongate member can further comprise a hinge extending along the longitudinal axis of the elongate member for the entire length of the elongate member in accordance with the first embodiment of the disclosure. Although the hinge is described as extending along the entire length of the elongate member in an embodiment, the hinge can extend along only a portion thereof or alternatively, a plurality of hinges can be utilized in a plurality of locations along the length of the elongate member in other embodiments. The hinge can be disposed at a second circumferential location of the elongate member that is substantially about 180 degrees from the first circumferential location. In accordance with some embodiments of the disclosure, the hinge comprises a living hinge. In accordance with the first embodiment of the disclosure, the first configuration can correspond to when the hinge is in an open position, and the second configuration can correspond to when the hinge is in a closed position. The elongate member can further comprise a first connection member or formation and a second connection member or formation configured to cooperate with the first connection member or formation so as to releasably lock the catheter insertion device in the second configuration in some embodiments of the disclosure. For example and without limitation, the first connection member or formation can comprise a tab, and the second connection member or formation can comprise a clasp. The first and second connection members or formations can comprise a zipper element configured to close the slit and releasably lock the catheter insertion device in the second configuration in some embodiments of the disclosure. The elongate member can include a radially outwardly extending projection extending around a circumference of the catheter insertion device that is configured to releasably mate with a hemostasis valve in accordance with some embodiments of the disclosure.

In a second embodiment, the elongate member can comprise a first telescoping section, a second telescoping section, and at least one middle telescoping section telescopically connected between the first telescoping section and the second telescoping section. Adjacent telescoping sections can be slidable relative to one another between retracted and extended positions. In the second embodiment, the first configuration can correspond to a condition where the telescoping sections are in retracted positions, and the second configuration can correspond to a condition where the telescoping sections are in extended positions. The elongate member can further include a plurality of detents on each of the telescoping sections configured for engagement to releasably lock the catheter insertion device in the second configuration. The elongate member can further include a control system for displacing the telescoping sections between retracted and extended positions in accordance with some embodiments of the disclosure. The elongate member can include a radially outwardly extending projection extending around a circumference of the catheter insertion device that is configured to releasably mate with a hemostasis valve in accordance with some embodiments of the disclosure.

A medical kit is provided comprising a catheter configured for insertion within a hemostasis valve; and a catheter insertion device. The catheter insertion device may comprise any of the embodiments described above or hereinafter. For example, the catheter insertion device can comprises an elongate member configured to be disposed on the catheter to allow insertion of the catheter through the hemostasis valve. The catheter insertion device can be configured to be disposed on the catheter in a first configuration and a second configuration. The first configuration and the second configuration are different. The catheter insertion device can be further configured to allow the elongate member to be selectively and repeatedly reconfigured between the first configuration and the second configuration while disposed on the catheter. The catheter insertion device can be configured to allow a user (e.g., a physician) to repeatedly switch between the first configuration and the second configuration while disposed on the catheter. The catheter insertion device can also include a radially outwardly extending projection extending around a circumference of the catheter insertion device configured to releasably mate with the hemostasis valve.

In accordance with a first embodiment of the disclosure, the elongate member of the catheter insertion device of the medical kit can comprise a slit extending along the elongate member for an entire length of the elongate member and a living hinge extending along the longitudinal axis of the elongate member for the entire length of the elongate member. The slit can be disposed at a first circumferential location of the elongate member, and the hinge can be disposed at a second circumferential location of the elongate member that is substantially about 180 degrees from the first circumferential location. The elongate member can further include a first connection member or formation near the slit and a second connection member or formation near the slit that are configured for engagement to releasably lock the catheter insertion device in the second configuration.

In accordance with a second embodiment of the disclosure, the elongate member of the medical kit can comprise a first telescoping section; a second telescoping section; at least one middle telescoping section telescopically connected between the first telescoping section and the second telescoping section; and a plurality of detents on each of the telescoping sections configured for engagement to releasably lock the catheter insertion device in the second configuration. Adjacent telescoping sections are slidable relative to one another between retracted and extended positions. The medical kit can further comprise a control system for displacing the telescoping sections between retracted and extended positions.

A catheter insertion device is provided comprising an elongate member defining a longitudinal axis; means for disposing the elongate member on a catheter in a first configuration and a second configuration, wherein the first configuration and the second configuration are different, and for allowing the elongate member to be selectively and repeatedly reconfigured between the first configuration and the second configuration; and means for releasably locking the elongate member in at least one of the first configuration and the second configuration. The catheter insertion device can further comprise means for releasably mating the catheter insertion device with a hemostasis valve.

The foregoing and other aspects, features, details, utilities, and advantages of the present disclosure will be apparent from reading the following description and claims, and from reviewing the accompanying drawings.

### BRIEF DESCRIPTION OF THE DRAWINGS

FIG. 1 is a lateral elevational view of a catheter insertion device in accordance with a first embodiment of the disclosure and a hemostasis valve.

FIG. 2 is an enlarged view of a portion of the hemostasis valve of FIG. 1.

FIG. 3 is a lateral cross-sectional view of the catheter insertion device of FIG. 1.

FIG. 4A is an axial cross-sectional view of the catheter insertion device of FIGS. 1 and 3 in a first configuration taken along line 4A-4A of FIG. 3.

FIG. 4B is an axial cross-sectional view of the catheter insertion device of FIG. 1 in a second configuration.

FIG. 5 is a cross-sectional view of a portion of the hemostasis valve and catheter insertion device of FIG. 1.

FIG. 6A is an isometric view of a catheter insertion device in accordance with a second embodiment of the disclosure in a first configuration.

FIG. 6B is an isometric view of the catheter insertion device of FIG. 6A in a second configuration.

### DETAILED DESCRIPTION OF THE DISCLOSURE

Various embodiments are described herein to various apparatuses, systems, and/or methods. Numerous specific details are set forth to provide a thorough understanding of the overall structure, function, manufacture, and use of the embodiments as described in the specification and illustrated in the accompanying drawings. It will be understood by those skilled in the art, however, that the embodiments may be practiced without such specific details. In other instances, well-known operations, components, and elements have not been described in detail so as not to obscure the embodiments described in the specification. Those of ordinary skill in the art will understand that the embodiments described and illustrated herein are non-limiting examples, and thus it can be appreciated that the specific structural and functional details disclosed herein may be representative and do not necessarily limit the scope of the embodiments, the scope of which is defined solely by the appended claims.

Reference throughout the specification to “various embodiments,” “some embodiments,” “one embodiment,” or “an embodiment”, or the like, means that a particular feature, structure, or characteristic described in connection with the embodiment is included in at least one embodiment. Thus, appearances of the phrases “in various embodiments,” “in some embodiments,” “in one embodiment,” or “in an embodiment”, or the like, in places throughout the specification are not necessarily all referring to the same embodiment. Furthermore, the particular features, structures, or characteristics may be combined in any suitable manner in one or more embodiments. Thus, the particular features, structures, or characteristics illustrated or described in connection with one embodiment may be combined, in whole or in part, with the features structures, or characteristics of one or more other embodiments without limitation given that such combination is not illogical or non-functional.

It will be appreciated that the terms “proximal” and “distal” may be used throughout the specification with reference to a clinician manipulating one end of an instrument used to treat a patient. The term “proximal” refers to the portion of the instrument closest to the clinician and the term “distal” refers to the portion located furthest from the clinician. It will be further appreciated that for conciseness and clarity, spatial terms such as “vertical,” “horizontal,” “up,” and “down” may be used herein with respect to the illustrated embodiments. However, surgical instruments may be used in many orientations and positions, and these terms are not intended to be limiting and absolute.

The instant disclosure generally relates to catheter insertion devices. For purposes of this description, identical or similar aspects among the various embodiments described herein will be referred to by identical or similar reference numbers. As will be appreciated, however, the structure of the various aspects can be different among the various embodiments.

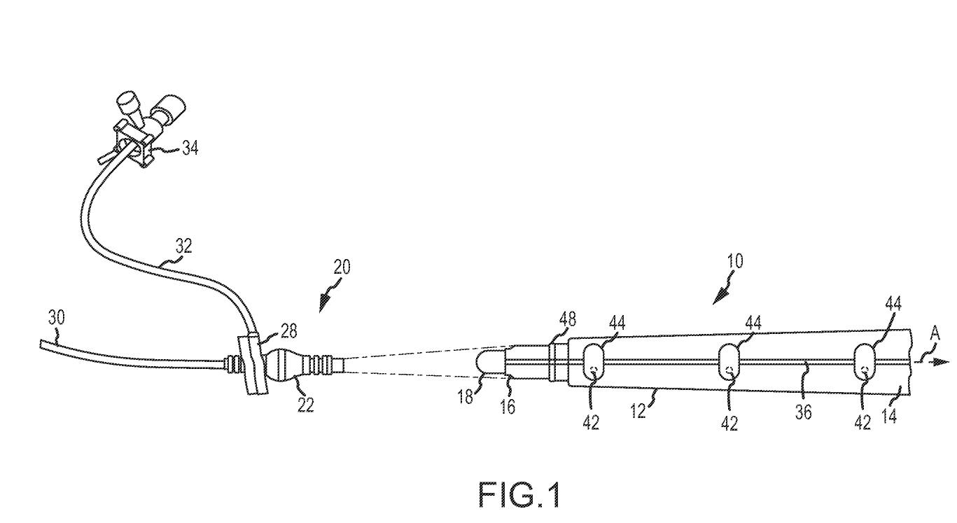

Referring now to FIG. 1, a lateral elevational view of a catheter insertion device 10 in accordance with a first embodiment of the disclosure is generally illustrated. The catheter insertion device 10 comprises an elongate, tubular member 12 compatible with blood and other body fluids/tissue. The elongate member 12 defines a longitudinal axis (A). The elongate member 12 can be flexible. However, the elongate member 12 can have sufficient rigidity to facilitate insertion of the elongate member 12 into a hemostasis valve 20. The elongate member 12 can comprise a biocompatible polymer material in accordance with some embodiments of the disclosure. For example and without limitation, the elongate member 12 can comprise high density polyethylene (HDPE). The elongate member 12 can have a proximal end 14 and a distal end 16 and a lumen extending therethough. In an embodiment and without limitation, the elongate member 12 can be about 3.7 inches (about 9.4 cm) in overall length from proximal end 14 to distal end 16, have a first diameter at the proximal end 14 of about 0.25 inches (about 0.6 cm), and have a second diameter at the distal end 16 of about 0.125 inches (about 0.3 cm). Although these particular dimensions are mentioned in detail, the dimensions for the elongate member 12 for the catheter insertion device 10 can vary in accordance with various embodiments of the disclosure. For example, in one embodiment, the elongate member 12 can have a substantially constant inner and/or outer diameter along its entire length. Although this particular configuration is mentioned in detail, the configuration for the elongate member 12 for the catheter insertion device 10 can vary in accordance with various embodiments of the disclosure. For example and without limitation, the elongate member 12 can be tapered along a portion thereof and have a stepped inner and/or outer diameter design along a portion thereof in accordance with some embodiments of the disclosure.

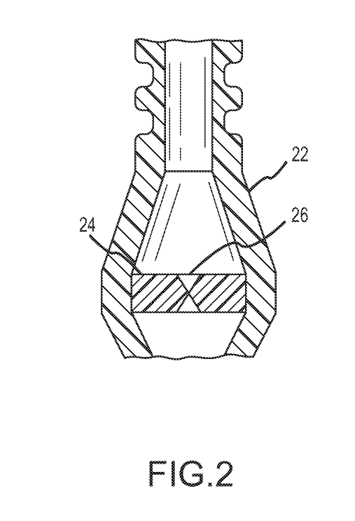

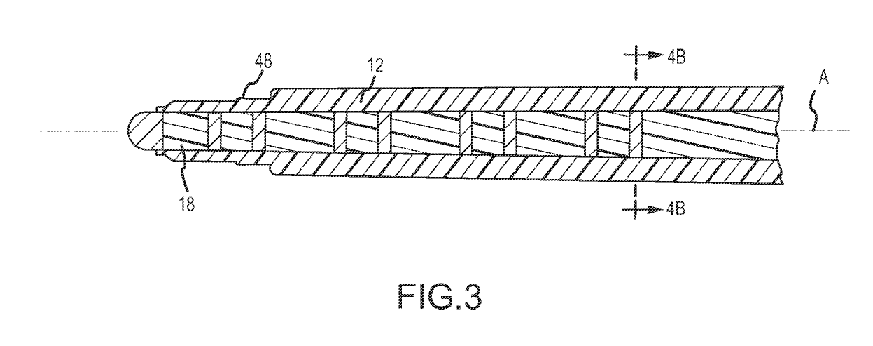

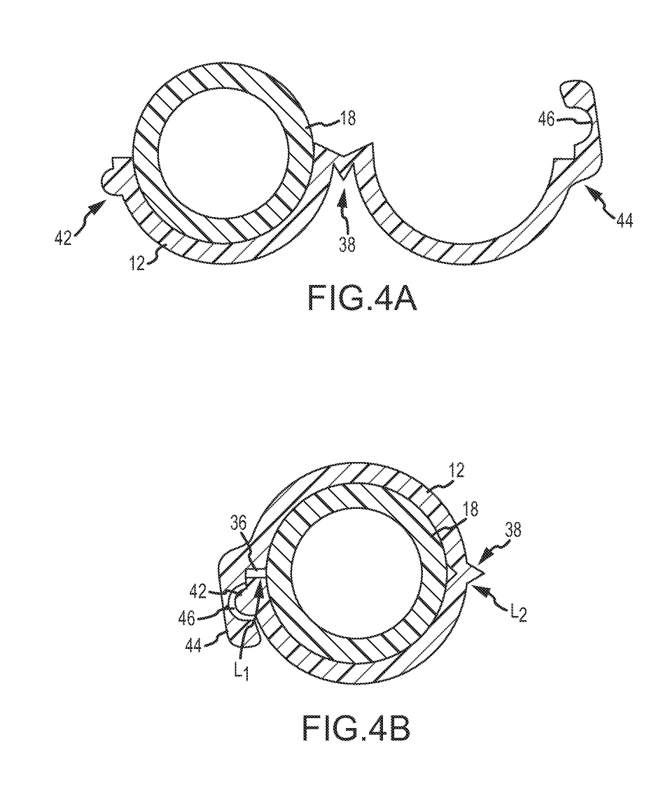

Still referring to FIG. 1, the elongate member 12 is configured to be disposed so as to surround a distal portion of a catheter 18 when in a closed configuration to be described below. The catheter 18 can be a curved, circular, or looped catheter in accordance with various embodiments of the disclosure. For example and without limitation, the elongate member 12 may externally surround or circumscribe the catheter 18 as best illustrated in FIGS. 1 and 3. The elongate member 12 may completely circumscribe at least a portion of the catheter 18 in at least one configuration in accordance with some embodiments of the disclosure, such that the entire circumference of the catheter 18 is covered by the elongate member 12 when the elongate member 12 is in at least one configuration as generally illustrated by FIG. 4B. Referring to FIG. 1, the elongate member 12 and catheter 18 can be configured for insertion into a hemostasis valve 20. The hemostasis valve 20 can comprise a hub 22 molded from plastic. Contained within the hub 22 may be an elastomeric disk or membrane 24 (see FIG. 2) having a self-closing aperture 26 formed through a thickness dimension thereof. The hub 22 can further include a side port 28 having a bore (not shown) that is fluid communication with a lumen of an introducer 30. Introducer 30 can be a catheter or sheath in accordance with various embodiments of the disclosure. A length of tubing 32 may be connected to the side port 28. Affixed to the length of tubing 32 may be a stop cock member 34 that is conventional and known to those of ordinary skill in the art. Although an exemplary hemostasis valve 20 is generally described and illustrated, any number of hemostasis valves 20 may be utilized in connection with the catheter insertion device 10 in accordance with various embodiments of the disclosure. An exemplary hemostasis valve can be similar to the hemostasis valve described in U.S. Pat. No. 6,632,200 entitled “Hemostasis Valve,” which is hereby incorporated by reference as though fully set forth herein.

Referring now to FIG. 4B, the catheter insertion device 10 further comprises a means (best shown in FIG. 4B) for selectively disposing the elongate member 12 in a first configuration or a second configuration, and for allowing the elongate member 12 to be repeatedly reconfigured between the first configuration and the second configuration. For example and without limitation, the means for disposing the elongate member 12 in a first configuration and a second configuration and allowing the elongate member 12 to be repeatedly reconfigured between the first configuration and the second configuration may comprise a slit 36 and a hinge 38. As best illustrated in FIGS. 1 and 4B, the elongate member 12 comprises slit 36. The slit 36 may extend parallel to or along the longitudinal axis (A) of the elongate member 12. The slit 36 may extend for an entire length of the elongate member 12 from proximal end 14 to distal end 16 in accordance with embodiments of the disclosure. As best illustrated in FIG. 4B, the slit 36 can be disposed at a first circumferential location (L₁) of the elongate member 12 along the circumference of elongate member 12. Although means for disposing the elongate member 12 in a first configuration and a second configuration is noted as comprising a slit 36 and hinge 38, the means for disposing the elongate member in a first configuration and a second configuration can comprise any number of other mechanisms, such as the elongate member 12 comprising two separate portions or halves that may be unconnected in a first configuration, but when combined are configured to circumferentially surround the catheter 18 in a second configuration.

Referring now to FIGS. 4A-4B, the elongate member 12 further comprises a hinge 38. The hinge 38 can be configured to extend along the longitudinal axis (A) of the elongate member 12, and the hinge 38 can be configured to extend for an entire length of the elongate member 12 from proximal end 14 to distal end 16 in accordance with some embodiments of the disclosure. As best illustrated in FIG. 4B, the hinge 38 can be disposed at a second circumferential location (L₂) of the elongate member 12 along the circumference of elongate member 12. The second circumferential location (L₂) can be located about 180 degrees from the first circumferential location (L₁) in accordance with some embodiments of the disclosure. Although the second circumferential location (L₂) is described and illustrated as being located about 180 degrees from the first circumferential location (L₁), the second circumferential location (L₂) may be located closer or farther from the first circumferential location (L₁) in accordance with various embodiments of the disclosure. The hinge 38 can comprise a living hinge 18 in accordance with some embodiments of the disclosure. The living hinge 38 may comprise a thin, flexible section of material, allowing for bending along a portion of the hinge 38. The living hinge 38 can be made from the same material as the remainder of the elongate member 12 in accordance with some embodiments of the disclosure. In accordance with other embodiments of the disclosure, the living hinge 38 can be made from a different material than the remainder of the elongate member 12. For example and without limitation, the living hinge 38 can be formed of a plastic having a lower durometer than the remainder of the elongate member 12, thereby resulting in the living hinge 38 having more flexibility than the remainder of the elongate member 12. The living hinge 38 can be formed integrally with the remainder of the elongate member 12 as a single, one-piece, unitary, integral, monolithic structure. For example and without limitation, the elongate member 12 and living hinge 38 can be formed via extrusion and/or molding processes generally known to those of ordinary skill in the art. The hinge 38 may be configured to allow the catheter insertion device 10 to be opened and closed along its entire length.

The elongate member 12 is configured to be disposed on the catheter 18 in a first configuration as generally illustrated in FIG. 4A and a second configuration as generally illustrated in FIG. 4B. The first configuration and the second configuration are different. In particular, the first configuration generally illustrated in FIG. 4A can correspond to when the hinge 38 is in an open position. The second configuration generally illustrated in FIG. 4B can correspond to when the hinge 38 is in a closed position. The catheter insertion device 10 is configured to allow a user (e.g., a physician) to repeatedly and selectively switch between the first configuration and the second configuration while the catheter insertion device 10 is disposed at least partially on the catheter 18. In particular, the catheter insertion device 10 can be repeatedly switched between the first and second configurations and can be replaced and removed from the catheter 18 multiple times without compromising the integrity of the catheter insertion device 10. The elongate member 12 allows for straightening of curved or looped catheters 18, while being configured to be removed from the catheter 18 after positioning of the catheter 18 within the hemostasis valve 20. In this embodiment, when the elongate member 12 is removed from the catheter 18, the catheter 18 can have increased usable length of the shaft of the catheter 18 for various diagnostic, therapeutic, and/or mapping and ablation procedures.

The elongate member 12 can comprise a means for releasably locking the elongate member 12 in at least one of the first configuration and the second configuration. In particular, the means for releasably locking the elongate member 12 can be configured to releasably lock the elongate member 12 in the second configuration in which the hinge 38 is in a closed position so that the elongate member 12 can retain the catheter 18 without separating.

For example and without limitation, the means for releasably locking the elongate member 12 can comprise a first connection member 42 near the slit 36 and a second connection member 44 near the slit 36. The first connection member 42 can comprise a tab 42 in accordance with some embodiments of the disclosure. The second connection member 44 can comprise a clasp 44 configured to engage the tab 42 in accordance with some embodiments of the disclosure. For example and without limitation, the clasp 44 is configured for engagement with the tab 42 to releasably lock the catheter insertion device 10 in the second configuration in which the hinge 38 is in a closed position. For example and without limitation, the tab 42 can be configured for an interference fit or friction fit with a recess 46 in the clasp 44. In other words, the tab 42 may “snap” into the recess 46 in the clasp 44 in accordance with some embodiments of the disclosure. As generally illustrated in FIG. 1, the catheter insertion device 10 can include a plurality of first connection members 42 (shown in phantom) and second connection members 44 that are disposed along the length of the elongate member 12 from proximal end 14 to distal end 16. For example and without limitation, the first and second connection members 42, 44 may be substantially evenly spaced along the length of the elongate member 12. Although three pairs of connection members 42, 44 are illustrated in FIG. 1, there may be fewer or more first and second connection members 42, 44 and they may be arranged in any number of spacing arrangements in various embodiments of the disclosure.

Although a tab 42 and clasp 44 are described and illustrated in detail, the elongate member 12 of the catheter insertion device 10 can include any number of other means for releasably locking the elongate member 12 in at least one of the first configuration and the second configuration. For example and without limitation, the means for releasably locking the elongate member 12 may include at least one first connection member that is configured for elastic deformation in order to allow for engagement with a second connection member. After elastic deformation, the first connection member may then return to its original shape. For another example and without limitation, the means for releasably locking the elongate member 12 in at least one of the first configuration and the second configuration can comprise a zipper element as generally known to those of ordinary skill in the art configured to close the slit 36. The zipper element can extend longitudinally parallel to or along the axis (A) of the elongate member 12. The zipper element can be configured to releasably lock the catheter insertion device 10 along its entire length from proximal end 14 to distal end 16 and be configured for re-opening. For another example and without limitation, the means for releasably locking the elongate member 12 in at least one of the first configuration and the second configuration can comprise a zip lock closure element as generally known to those of ordinary skill in the art configured to close the slit 36. The zip lock closure element can extend longitudinally parallel to or along the axis (A) of the elongate member 12. The zip lock closure element can be configured to releasably lock the catheter insertion device 10 along its entire length from proximal end 14 to distal end 16 and be configured for re-opening.

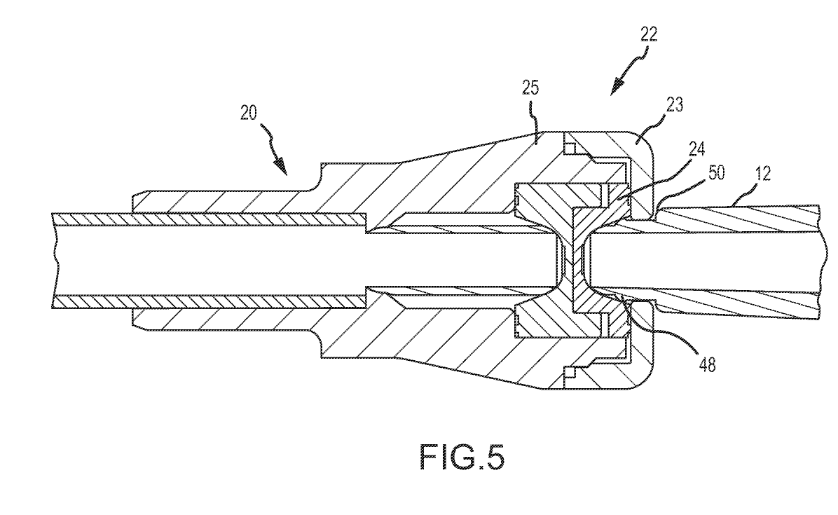

Referring now to FIGS. 1, 3, and 5, the catheter insertion device 10 can further include a means for releasably mating the catheter insertion device 10 to a hemostasis valve 20. The means for releasably mating the catheter insertion device 10 to a hemostasis valve 20 can comprise a radially outwardly extending projection 48 on an outer surface of the elongate member 12 in accordance with some embodiments of the disclosure. The radially outwardly extending projection 48 can extend around the circumference of the catheter insertion device 10 in accordance with an embodiment of the disclosure. The radially outwardly extending projection 48 can result in the elongate member 12 of the catheter insertion device 10 having an increased diameter at the point of the radially outwardly extending projection 48 relative to the remainder of the elongate member 12 near the projection 48. The projection 48 may be configured to releasably mate with or engage the hemostasis valve 20. In particular, the projection 48 may be configured to releasably lock the catheter insertion device 10 onto the hub 22 of the hemostasis valve 20 to aid in insertion of the catheter 18 into the hemostasis valve 20. As generally illustrated in FIG. 5, the hemostasis valve 20 can include a hub cap 23 welded to the hub body 25, thereby forming hub 22. The catheter insertion device 10 is configured to releasably mate with the hub cap 23 via the projection or raised portion 48 shown on the left of the wall of the hub cap 23 and a shoulder or mechanical stop 50 shown on the right of the wall of the hub cap 23 in FIG. 5. For example and without limitation, the catheter insertion device 10 is configured to elastically deform and/or “snap” into the hemostasis valve 20. The shoulder or mechanical stop 50 can be configured to limit travel of the catheter insertion device 10 into the hemostasis valve. The catheter insertion device 10 is configured to protrude through the elastomeric disk or membrane 24 to open it and allow for unrestricted passage of the catheter 18 through the hemostasis valve 20. While such protrusion may cause at least a portion of membrane 24 to deflect, such deflection is not shown in FIG. 5.

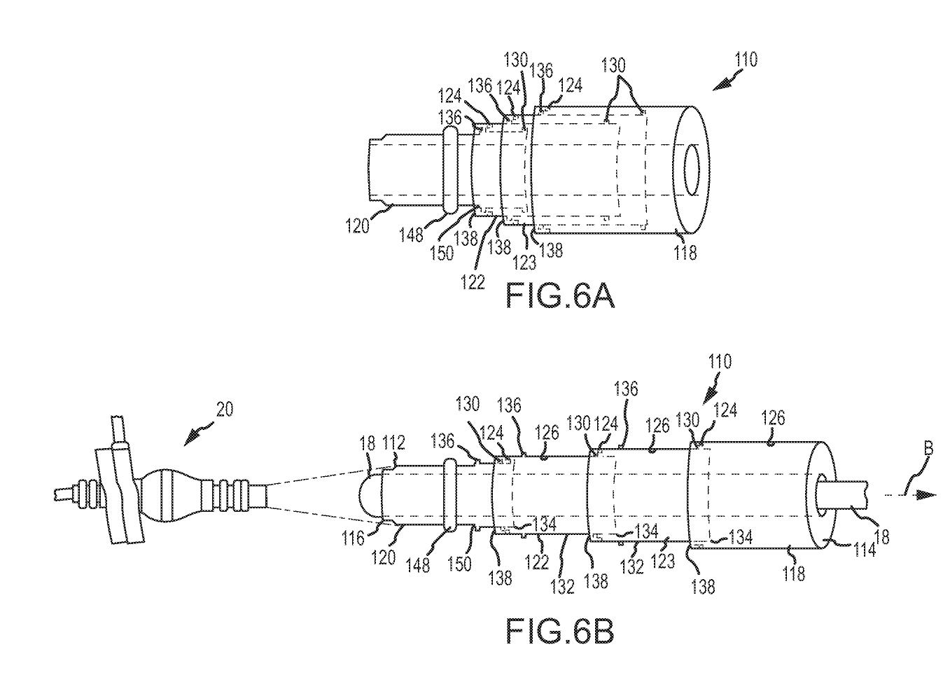

Referring now to FIGS. 6A-6B, a catheter insertion device 110 in accordance with a second embodiment of the disclosure is generally illustrated. The catheter insertion device 110 comprises an elongate, tubular member 112 compatible with blood and other body fluids/tissue. The elongate member 112 defines a longitudinal axis (B). The elongate member 112 can be flexible. However, the elongate member 112 can have sufficient rigidity to facilitate insertion of the elongate member 112 into a hemostasis valve 20. The elongate member 112 can comprise a biocompatible polymer material in accordance with some embodiments of the disclosure. For example and without limitation, the elongate member 112 can comprise HDPE. The elongate member 112 can have a proximal end 114 and a distal end 116 and a lumen extending therethough. The elongate member 112 can be about 0.75 to about 1.00 inches (about 1.9 to about 2.5 cm) in length from a proximal end 114 to a distal end 116 when the elongate member 112 is in a first configuration as described in more detail below and generally illustrated in FIG. 6A. The elongate member 112 can be about 3.5 inches (about 8.9 cm) in overall length from proximal end 114 to distal end 116 when the elongate member 112 is in a second configuration as described in more detail below and generally illustrated in FIG. 6B. The elongate member 112 can have a first diameter at the proximal end 114 of about 0.25 inches (about 0.6 cm) and a second diameter at the distal end 116 of about 0.125 inches (about 0.3 cm). Although these particular dimensions are mentioned in detail, the dimensions for the elongate member 112 for the catheter insertion device 110 can vary in accordance with various embodiments of the disclosure.

The catheter insertion device 110 further comprises a means (best shown in FIG. 6A) for disposing the elongate member 112 in one of a first configuration and a second configuration, and for allowing the elongate member 112 to be repeatedly reconfigured between the first configuration and the second configuration. For example and without limitation, the means for disposing the elongate member 112 in one of a first configuration and a second configuration comprises telescoping sections 118, 120, 122, 123 of elongate member 112 that are configured for movement relative to each other. In particular, the elongate member 112 comprises a first end telescoping section 118. The first end telescoping section 118 can be located at least partially at the proximal end 114 of the elongate member 112. The elongate member 112 further comprises a second end telescoping section 120. The second end telescoping section 120 can be located at least partially at the distal end 116 of the elongate member 112. The elongate member 112 further comprises at least one middle telescoping section 122, 123 telescopically connected between the first end telescoping section 118 and the second end telescoping section 120. Although two middle telescoping sections 122, 123 are generally illustrated, there may be fewer or more middle telescoping sections 122, 123 in accordance with various embodiments of the disclosure.

Adjacent telescoping sections 118, 120, 122, 123 can be slidable relative to one another between retracted and extended positions. In the retracted position, the first end telescoping section 118 is proximally slid over at least one middle telescoping section 122 and at least one middle telescoping section 122 is slid over the second end telescoping section 120. If there are multiple middle telescoping sections, then proximally located middle telescoping sections, such as section 123, are slid over adjacent distally located middle telescoping sections, such as section 122, to move to a retracted position. In the extended position, the first end telescoping section 118 is extended so that it does not substantially cover at least one middle telescoping section, such as section 123, and at least one middle telescoping section, such as section 122, is extended so that it does not substantially cover the second end telescoping section 120.

The elongate member 112 is configured to be selectively disposed on a catheter 18 in accordance with an embodiment of the disclosure. The catheter 18 can be a curved, circular, or looped catheter in accordance with various embodiments of the disclosure. The elongate member 112 and the catheter 18 can be configured for insertion into a hemostasis valve 20 as described hereinabove. The elongate member 112 is configured to be at least partially disposed on the catheter 18 in a first configuration as generally illustrated in FIG. 6A and a second configuration as generally illustrated in FIG. 6B. The first configuration and the second configuration are different. In particular, the first configuration generally illustrated in FIG. 6A can correspond to when at least one or each of the telescoping sections 118, 120, 122, 123 is in its respective retracted position wherein a proximally located telescoping section is slid over an adjacent distally located telescoping section. In the first configuration, the elongate member 112 may completely circumscribe a portion of the catheter 18 such that the entire circumference of the portion of the catheter 18 is covered by the elongate member 112. The portion of the catheter 18 that may be completely circumscribed may be only about 0.75 to about 1.00 inches (about 1.9 to about 2.5 cm) in length in accordance with an embodiment of the disclosure. The second configuration generally illustrated in FIG. 6B can correspond to when each of the telescoping sections 118, 120, 122, 123 is in its respective extended position wherein a proximally located telescoping section is extended so that it does not substantially cover an adjacent distally located telescoping section. In the second configuration, the elongate member 112 may also completely circumscribe a portion of the catheter 18 such that the entire circumference of the catheter 18 is covered by the elongate member 112. The portion of the catheter 18 that may be completely circumscribed may be about 3.5 inches (about 8.9 cm) in length in accordance with an embodiment of the disclosure. Accordingly, when the catheter insertion device 110 is in the first configuration, it may result in a shorter overall length of the catheter insertion device 110 that may be about ⅕ to about ⅓ of the length of the catheter insertion device 110 in the second configuration. Although particular dimensions and configurations are mentioned in detail, the dimensions and configuration may vary in accordance with other embodiments of the disclosure.

The catheter insertion device 110 is configured to allow a user (e.g., a physician) to selectively and repeatedly switch between the first configuration and the second configuration while the catheter insertion device 110 is at least partially disposed on the catheter 18. In particular, the catheter insertion device 110 can be repeatedly switched between the first and second configurations (e.g., between the retracted and extended positions of the telescoping sections 118, 120, 122, 123) multiple times without compromising the integrity of the catheter insertion device 110. The elongate member 112 allows for straightening of curved or looped catheters 18 while the elongate member 12 is in the second configuration of FIG. 6B, while being configured to be retracted in the first configuration of FIG. 6A after positioning of the catheter 18 within the hemostasis valve 20 to make additional usable length of the catheter 18 available. When the elongate member 112 is retracted such that the telescoping positions 118, 120, 122 are each in a retracted position such as generally illustrated in FIG. 6A, the catheter 18 can have increased usable length of the shaft of the catheter 18 for various diagnostic, therapeutic, and/or mapping and ablation procedures. Additionally, the catheter insertion device 110 can include a control system for displacing the telescoping sections 118, 120, 122, 123 between retracted and extended positions. For example and without limitation, the control system may comprise a compressor or other pneumatic control mechanism. Although this control system is mentioned in detail, any number of other control systems known to those of ordinary skill in the art can be utilized in various embodiments of the disclosure.

The elongate member 112 can comprise a means for releasably locking the elongate member 112 in at least one of the first configuration and the second configuration. In particular, the means for releasably locking the elongate member 112 can be configured to releasably lock the elongate member 112 in the second configuration in which one or more or each of the telescoping sections 118, 120, 122, 123 is in its respective extended position wherein a proximally located telescoping section is extended so that it does not substantially cover an adjacent distally located telescoping section. The means for releasably locking the elongate member 112 can also be configured to releasably lock the elongate member 112 in the first configuration in which one or more or each of the telescoping sections 118, 120, 122, 123 is in its respective retracted position wherein a proximally located telescoping section is slid over an adjacent distally located telescoping section.

For example and without limitation, the means for releasably locking the elongate member 112 can comprise a plurality of detents located on one or more or each of the telescoping sections 118, 120, 122, 123. For example and without limitation, the first end telescoping section 118 and each of the middle telescoping sections 122, 123 can include a first plurality of detents 124 on an inner radial surface 126 of a distal end 138 of each of the telescoping sections 118, 122, 123. The second end telescoping section 120 and each of the middle telescoping sections 122, 123 can include a second plurality of detents 130 on an outer radial surface 132 of a proximal end 134 of each of the telescoping sections 120, 122, 123. The first plurality of detents 124 and the second plurality of detents 130 are configured for engagement to releasably lock the catheter insertion device 110 in the second configuration as generally illustrated in FIG. 6B.

In addition, the second end telescoping section 120 and each of the middle telescoping sections 122, 123 can include a third plurality of detents 136 on the outer radial surface 132 near a distal end 138 of each of the telescoping sections 120, 122, 123. The first plurality of detents 124 and the third plurality of detents 136 are configured for engagement to releasably lock the catheter insertion device 110 in the first configuration as generally illustrated in FIG. 6A. Although detents 124, 130, 136 are described and illustrated in detail, the means for releasably locking the elongate member 112 can comprise any number of other connection members, formations, and/or engagement elements known to those of ordinary skill in the art. For example and without limitation, snap elements may be used to lock the elongate member 112 in the first configuration and/or second configuration.

Still referring to FIGS. 6A-6B, the catheter insertion device 110 can further include a means for releasably mating the catheter insertion device 110 to a hemostasis valve 20. The means for releasably mating the catheter insertion device 110 to a hemostasis valve 20 can comprise a radially outwardly extending projection 148 on an outer surface of the elongate member 112 in accordance with some embodiments of the disclosure. In at least one embodiment, the catheter insertion device 110 can further include the radially outwardly extending projection 148 on the outer surface of the elongate member 112. The radially outwardly extending projection 148 can extend around the circumference of the catheter insertion device 110 in accordance with an embodiment of the disclosure. The radially outwardly extending projection 148 can result in the elongate member 112 of the catheter insertion device 110 having an increased diameter at the point of the radially outwardly extending projection 148 relative to the remainder of the elongate member 112 near the projection 148. The projection 148 is configured to releasably mate with or engage the hemostasis valve 20. In particular, the projection 148 is configured to releasably lock the catheter insertion device 10 onto the hub 22 of the hemostasis valve 20 to aid in insertion of the catheter 18 into the hemostasis valve 20. The catheter insertion device 110 is configured to releasably mate with the hub cap 23 (similar to that shown in FIG. 5) via the projection or raised portion 148 and a shoulder or mechanical stop 150. The shoulder or mechanical stop 150 can be configured to limit travel of the catheter insertion device 110 into the hemostasis valve. For example and without limitation, the catheter insertion device 110 can be configured to elastically deform and/or “snap” into the hemostasis valve 20. The catheter insertion device 110 is configured to protrude through the elastomeric disk or membrane 24 (similar to that shown in FIG. 5) to open it and allow for unrestricted passage of the catheter 18 through the hemostasis valve 20.

Although at least two embodiments of this disclosure have been described above with a certain degree of particularity, those skilled in the art could make numerous alterations to the disclosed embodiments without departing from the spirit or scope of this disclosure. All directional references (e.g., upper, lower, upward, downward, left, right, leftward, rightward, top, bottom, above, below, vertical, horizontal, clockwise, and counterclockwise) are only used for identification purposes to aid the reader's understanding of the present disclosure, and do not create limitations, particularly as to the position, orientation, or use of the disclosure. Joinder references (e.g., attached, coupled, connected, and the like) are to be construed broadly and can include intermediate members between a connection of elements and relative movement between elements. As such, joinder references do not necessarily infer that two elements are directly connected and in fixed relation to each other. It is intended that all matter contained in the above description or shown in the accompanying drawings shall be interpreted as illustrative only and not limiting. Changes in detail or structure can be made without departing from the spirit of the disclosure as defined in the appended claims.

Any patent, publication, or other disclosure material, in whole or in part, that is said to be incorporated by reference herein is incorporated herein only to the extent that the incorporated materials does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

What is claimed is:

1. A catheter insertion device comprising:
an elongate member defining a longitudinal axis, wherein the elongate member is configured to be disposed on a catheter in one of a first configuration and a second configuration, wherein the first configuration and the second configuration are different, and the device is further configured to allow the elongate member to be selectively and repeatedly reconfigured between the first configuration and the second configuration while at least partially disposed on the catheter.

2. The device of claim 1, wherein the elongate member comprises a slit extending along the elongate member for an entire length of the elongate member, and wherein the slit is disposed at a first circumferential location of the elongate member.

3. The device of claim 2, wherein the elongate member further comprises a hinge extending along the longitudinal axis of the elongate member for the entire length of the elongate member, and wherein the hinge is disposed at a second circumferential location of the elongate member.

4. The device of claim 3, wherein the second circumferential location is substantially about 180 degrees from the first circumferential location.

5. The device of claim 3, wherein the hinge comprises a living hinge.

6. The device of claim 3, wherein the first configuration corresponds to when the hinge is in an open position and wherein the second configuration corresponds to when the hinge is in a closed position.

7. The device of claim 3, wherein the elongate member further comprises a first connection member near the slit and a second connection member near the slit, wherein the first connection member and the second connection member are configured for engagement to releasably lock the catheter insertion device in the second configuration.

8. The device of claim 7, wherein the first connection member comprises a tab, and the second connection member comprises a clasp.

9. The device of claim 3, wherein the elongate member further comprises a zipper element configured to close the slit and releasably lock the catheter insertion device in the second configuration.

10. The device of claim 1, further comprising a radially outwardly extending projection extending around a circumference of the catheter insertion device configured to releasably mate with a hemostasis valve.

11. The device of claim 1, wherein the elongate member comprises a first telescoping section, a second telescoping section, and at least one middle telescoping section telescopically connected between the first telescoping section and the second telescoping section, wherein adjacent telescoping sections are slidable relative to one another between retracted and extended positions.

12. The device of claim 11, wherein the first configuration corresponds to when each of the telescoping sections is in its respective retracted position and the second configuration corresponds to when each of the telescoping sections is in its respective extended position.

13. The device of claim 11, further comprising:
a plurality of detents on each of the telescoping sections configured for engagement to releasably lock the catheter insertion device in the second configuration.

14. The device of claim 11, further comprising a control system for displacing the telescoping sections between retracted and extended positions.

15. A medical kit comprising:
a catheter configured for insertion within a hemostasis valve; and
a catheter insertion device comprising an elongate member defining a longitudinal axis, wherein the elongate member is configured to be disposed on the catheter to allow insertion of the catheter through the hemostasis valve, wherein the catheter insertion device is configured to be selectively disposed on the catheter in one of a first configuration and a second configuration, the device further configured to allow the elongate member to be repeatedly reconfigured between the first configuration and the second configuration while at least partially disposed on the catheter.

16. The medical kit of claim 15, wherein the elongate member comprises:
a slit extending along the elongate member for an entire length of the elongate member, wherein the slit is disposed at a first circumferential location of the elongate member;
a living hinge extending along the longitudinal axis of the elongate member for the entire length of the elongate member, wherein the hinge is disposed at a second circumferential location of the elongate member that is substantially about 180 degrees from the first circumferential location; and
a first connection member near the slit and a second connection member near the slit, wherein the first connection member and the second connection member are configured for engagement to releasably lock the catheter insertion device in the second configuration.

17. The medical kit of claim 15, wherein the elongate member comprises:
a first telescoping section;
a second telescoping section;
at least one middle telescoping section telescopically connected between the first telescoping section and the second telescoping section, wherein adjacent telescoping sections are slidable relative to one another between retracted and extended positions; and
a plurality of detents on each of the telescoping sections configured for engagement to releasably lock the catheter insertion device in the second configuration.

18. The medical kit of claim 15, further comprising a radially outwardly extending projection extending around a circumference of the catheter insertion device configured to releasably mate with the hemostasis valve.

19. A catheter insertion device comprising:
an elongate member defining a longitudinal axis;
means for disposing the elongate member on a catheter in a first configuration and a second configuration, wherein the first configuration and the second configuration are different, and for allowing the elongate member to be selectively and repeatedly reconfigured between the first configuration and the second configuration; and
means for releasably locking the elongate member in at least one of the first configuration and the second configuration.

20. The device of claim 19, further comprising means for releasably mating the catheter insertion device with a hemostasis valve.
